(19) United States
(12) Patent Application Publication
(10) Pub. No.: US 2019/0191968 A1
(43) Pub. Date: Jun. 27, 2019

(54) ENDOSCOPE

(71) Applicant: Olympus Corporation, Tokyo (JP)

(72) Inventor: Masayo Tsumaru, Sagamihara-shi (JP)

(73) Assignee: Olympus Corporation, Tokyo (JP)

(21) Appl. No.: 16/291,524

(22) Filed: Mar. 4, 2019

Related U.S. Application Data
(63) Continuation of application No. PCT/JP2017/011951, filed on Mar. 24, 2017.

(30) Foreign Application Priority Data
Sep. 5, 2016 (JP) 2016-172970

(51) Int. Cl. A61B 1/00; A61B 1/12; G02B 23/24

(52) U.S. Cl. CPC A61B 1/00057; A61B 1/123; G02B 23/2476; A61B 1/00142; A61B 1/00066

(57) ABSTRACT

The disclosed technology is directed to an endoscope comprises a first adhesive layer disposed on a surface of an outer cladding member and having at least a portion exposed outwardly. A display portion includes a second adhesive layer disposed on a portion of the outer cladding member which is covered with the first adhesive layer. The second adhesive layer having at least a portion which is to be exposed outwardly deteriorates due to frequent cleaning, disinfecting, and/or sterilizing. The second adhesive includes a color different from the first adhesive layer. The display portion is defined by a color that is different from a color of the outer cladding member. The display portion includes characters or signs made of the second adhesive layer.

### CROSS-REFERENCE TO RELATED APPLICATIONS

This application is a continuation application of PCT Application No. PCT/JP2017/011951 filed on Mar. 24, 2017, which in turn claim priority to the Japanese Patent Application No. 2016-172970 filed on Sep. 5, 2016 in Japan which is hereby incorporated by reference in its entirety.

### TECHNICAL FIELD

The disclosed technology relates to an endoscope having an adhesive-securing portion on an outer cladding thereof.

### DESCRIPTION OF THE RELATED ART

In recent years, endoscopes have been used in the medical field, the industrial field, and so on. An endoscope includes an insertion portion that has an observation window and an illumination window on a distal-end portion thereof. The observation window and the illumination window are secured in place by an adhesive-securing portion that is coated with an adhesive in a predetermined region on a distal-end cover of the distal-end portion.

Some endoscopes have a bendable portion in an insertion portion thereof. The bendable portion includes an outermost layer made of bendable rubber that is secured in place by thread-wound adhesive portions, for example, disposed in a predetermined region on a distal-end hard member of the distal-end portion and a predetermined region on a joint tube disposed on a distal-end side of a flexible tube.

Endoscopes used in the medical field are cleaned and sterilized after being used for observation, diagnosis, treatment, or the like. The endoscopes are cleaned and sterilized by autoclaves or antiseptic solutions. The adhesive portions referred to hereinbefore are deteriorated gradually over time by being exposed to a water steam under high pressure and at high temperature in autoclave sterilization or by being immersed in antiseptic solutions.

Japanese Patent Laid-Open No. 1999(H11)-056746 discloses an endoscopic apparatus that prevents an endoscope from being broken by notifying the user before the endoscope deteriorates fatally. The disclosed endoscopic apparatus checks the number of times that the endoscope has been processed by cleaning, disinfecting, or sterilizing means, making it possible to repair the endoscope before it suffers a fatal failure such as a water leakage or the like thereby to increase the service life of the endoscope.

However, in a case where the number of times that the endoscope has been processed by a cleaning, disinfecting, or sterilizing device is used as a criterion for deterioration, as disclosed in Japanese Patent Laid-Open No. 1999(H11)-056746, the number of times as the criterion for deterioration varies under different sterilizing conditions such as types of chemicals used. Therefore, there have been demands from users for endoscopes that are capable of determining that an adhesive portion thereof has deteriorated to the extent that it needs repairing.

### BRIEF SUMMARY OF EMBODIMENTS

The disclosed technology has been made in view of the above circumstances. It is an object of the disclosed technology to provide an endoscope that is capable of easily determining how much an adhesive layer has deteriorated and capable of accurately determining whether the endoscope is to be sent for repair or not.

Accordingly, the disclosed technology is directed to an endoscope comprises a first adhesive layer disposed on a surface of an outer cladding member and having at least a portion exposed outwardly. A display portion includes a second adhesive layer disposed on a portion of the outer cladding member which is covered with the first adhesive layer. The second adhesive layer having at least a portion which is to be exposed outwardly deteriorates due to frequent cleaning, disinfecting, and/or sterilizing. The second adhesive includes a color different from the first adhesive layer. The display portion is defined by a color that is different from a color of the outer cladding member. The display portion includes characters or signs made of the second adhesive layer.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrative of an endoscope.

FIG. 2A is a view mainly illustrative of a distal-end face of a distal-end portion of an insertion portion.

FIG. 2B is a cross-sectional view taken along a line Y2B-Y2B of FIG. 2A, illustrative of an adhesive-securing portion by which an observation window is secured to a distal-end cover.

FIG. 3A is a view illustrative of the insertion portion that includes bendable rubber.

FIG. 3B is an enlarged view illustrating a portion indicated by an arrow Y3B of FIG. 3A, illustrative of a thread-wound adhesive-securing portion.

FIG. 4A is a view illustrative of an adhesive layer on an endoscope connector that a universal cord has on an end portion thereof.

FIG. 4B is a view illustrative of a character portion as a display portion to be covered and concealed by the adhesive layer.

FIG. 5A is a view illustrative of a determining colored portion as an indicator disposed on the endoscope connector.

FIG. 5B is a view illustrative of determining colored portions disposed adjacent to thread-wound adhesive-securing portions of the bendable rubber.

FIG. 6 is a view illustrative of a cleaning and disinfecting device for cleaning the endoscope.

FIG. 7 is a view illustrative of a manner in which an endoscope holding grating with the endoscope installed thereon is disposed in a cleaning tank.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

An embodiment of the disclosed technology will hereinafter be described with reference to the drawings.

In each of the figures used in the description that follows, some of the components are drawn to different scales in order to illustrate themselves in sizes large enough to be recognized in the figures. The disclosed technology should not be limited only to the numbers, shapes, size proportions, and relative positional relationships of the components illustrated in the figures.

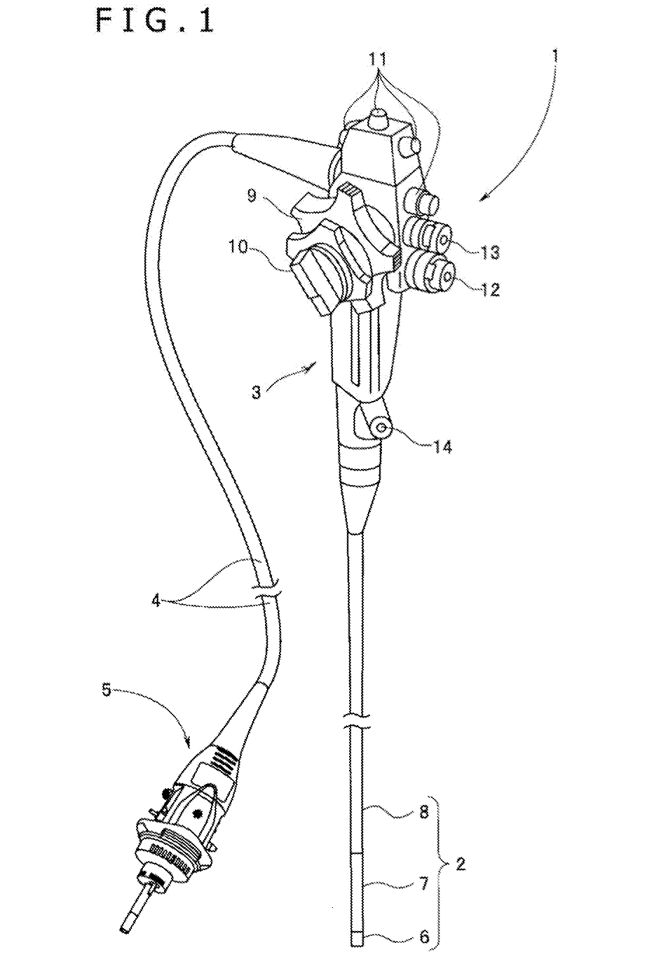

As illustrated in FIG. 1, an endoscope 1 mainly includes an insertion portion 2, a manipulator 3, and a universal cord 4. The insertion portion 2 is slender and flexible. The manipulator 3 is joined to a proximal-end side of the insertion portion 2 and is used as a grip. The universal cord 4 extends from a side of the manipulator 3. The universal cord 4 has on an end thereof an endoscope connector 5 for connection to a light source device, not illustrated.

The insertion portion 2 has a distal-end portion 6, a bendable portion 7 that is bendable, and a pliable flexible conduit 8 that is flexible, which are joined together successively from a distal-end side of the insertion portion 2. The manipulator 3 has bend manipulation levers 9 and 10 and remote switches 11, etc. disposed thereon. The remote switches 11 are switches for giving image control instructions such as freeze, release, etc. The bendable portion 7 is bent as the bend manipulation levers 9 and 10 is turned.

The manipulator 3 also has an air/water delivery button 12, an aspiration button 13, and a treatment tool insertion mouthpiece 14 mounted thereon. A forceps plug, not illustrated, is removably mounted on the treatment tool insertion mouthpiece 14.

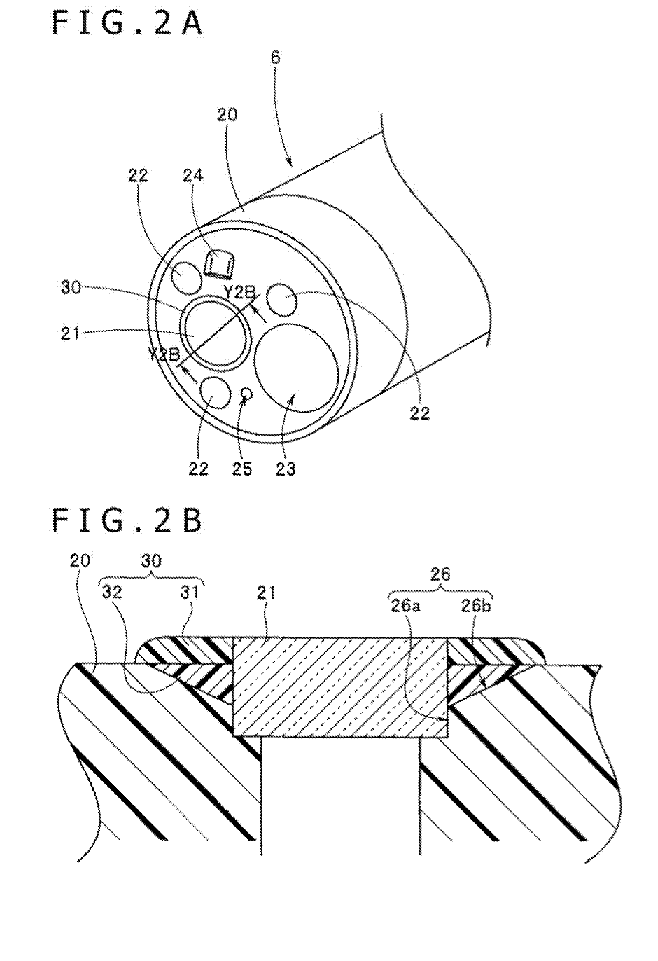

As illustrated in FIG. 2A, a distal-end cover member 20 is mounted on the distal-end portion 6. The distal-end cover member 20 is made of an insulative resin and is an outer cladding member that provides an outer cladding. The distal-end cover member 20 has an observation window 21, three illumination windows 22, for example, an aspiration opening 23 used as a treatment tool outlet tool, a nozzle 24, a forward water delivery port 25, or the like.

According to the present embodiment, the observation window 21 is secured to the distal-end cover member 20 by an adhesive-securing portion 30. According to the present embodiment, the adhesive-securing portion 30 has a determining function to determine deterioration of an adhesive, and functions as a securing portion and a determining portion.

As illustrated in FIG. 2B, the distal-end cover member 20 has an observation window installation hole 26 defined therein which has a window placement hole 26a and an adhesive coating space 26b. The window placement hole 26a is of a diameter slightly larger than an outside diameter of the observation window 21 and has a predetermined depth dimension.

The adhesive coating space 26b is a tapered hole and has a center aligned with the central axis of the window placement hole 26a. The adhesive coating space 26b has an outside diameter that is larger than an inside diameter of the window placement hole 26a by a predetermined dimension.

The observation window 21 is placed in the window placement hole 26a. The observation window 21 thus placed has a surface held in abutment against a bottom surface of the window placement hole 26a and the other surface, opposite the surface referred to hereinbefore, protruding from a distal-end surface of the distal-end cover member 20 by a predetermined distance.

A manner in which the observation window 21 is secured in the observation window installation hole 26 will be described hereinafter.

A worker places the observation window 21 in a predetermined state in the window placement hole 26a of the observation window installation hole 26. Then, the worker puts a second adhesive into the adhesive coating space 26b. The worker then planarizes a surface of a second adhesive layer 32 on the side of a distal-end cover surface that is exposed outwardly, causing the surface to lie substantially flush with the distal-end surface of the distal-end cover member 20. Thereafter, the second adhesive is hardened, securing the observation window 21 in the observation window installation hole 26.

After the second adhesive has been hardened, the worker applies a first adhesive to cover and conceal the surface of the second adhesive layer 32. Then, the worker, while keeping the surface of the second adhesive layer 32 concealed, planarizes a surface of a first adhesive layer 31 on the side of the other surface of the observation window 21, adjusting the first adhesive layer 31 to a predetermined thickness. According to the present embodiment, the planarized surface of the first adhesive layer 31 and the window distal-end surface provided by the other surface of the observation window 21 lie substantially flush with each other.

Thereafter, the first adhesive is hardened to secure the observation window 21 to the distal-end cover member 20. The adhesive-securing portion 30 is made up of the first adhesive layer 31 having an upper layer and having a thickness that is substantially uniform, the first adhesive layer 31 having an exposed surface, and the second adhesive layer 32 concealed by the first adhesive layer 31.

The first adhesive and the second adhesive referred to hereinbefore are different adhesives. According to the present embodiment, the first adhesive and the second adhesive have different colors. The first adhesive is black, whereas the second adhesive is bright orange. The first adhesive and the second adhesive have the same function as an adhesive.

As described hereinbefore, the adhesive-securing portion 30 disposed on the distal-end cover member 20 on the distal-end portion 6 of the endoscope 1 has the black first adhesive layer 31 and the orange second adhesive layer 32.

When the endoscope 1 is repeatedly cleaned and disinfected, the first adhesive layer 31, which is exposed outwardly, of the adhesive-securing portion 30 is caused to deteriorate. The first adhesive layer 31 is gradually worn away as it deteriorates, and as it is worn away, at least part of the orange second adhesive layer 32 is exposed through the worn-away area of the first adhesive layer 31.

The user now can judge that the first adhesive layer 31 has deteriorated by seeing the bright orange color, and recognize quickly without doubt that the timing to repair the endoscope 1 has come close. As a result, the user is able to prevent in advance the endoscope 1 from failing due to the deterioration of the adhesive in the endoscope 1 and to reliably send the endoscope 1 for repair without making any errors about the timing to repair the endoscope 1.

In the embodiment described hereinbefore, the second adhesive layer 32 has a predetermined function as an adhesive layer. Therefore, when the second adhesive layer 32 is exposed, it is not liable to be immediately responsible for a failure of the endoscope 1. In other words, the second adhesive layer 32 sufficiently performs its function as an adhesive during a predetermined period after it has been exposed.

Furthermore, in the embodiment described hereinbefore, the first adhesive and the second adhesive have the same function as an adhesive, but have different colors. However, the first adhesive and the second adhesive may have different functions. Specifically, the first adhesive is the least resistant to antiseptic solutions or a water steam under high pressure and at high temperature, among the adhesives used in other regions of the endoscope 1.

When the endoscope 1 is repeatedly cleaned and disinfected, therefore, the first adhesive layer 31 is caused to deteriorate earliest among the adhesive portions included in the endoscope 1. Consequently, the user is able to prevent in advance the endoscope 1 from failing due to the deterioration of the other adhesive portions and to reliably send the endoscope 1 for repair without making any errors about the timing to repair the endoscope 1.

Moreover, the color of the first adhesive should preferably be a color corresponding to the distal-end portion 6 of the endoscope 1. On the other hand, the color of the second adhesive should preferably be a color that is different from the color of the outer cladding member and is conspicuous and bright against the surroundings. The color of the second adhesive is not limited to the bright orange color.

Furthermore, in the embodiment described hereinbefore, the adhesive is of a double-layer structure in order to allow the adhesive-securing portion 30 that secures the observation window 21 to have the function to determine the deterioration of the adhesive. However, an adhesive portion, not illustrated, that secures the illumination window 22 to the distal-end cover member 20 may include an adhesive-securing portion functioning as a securing portion and a determining portion.

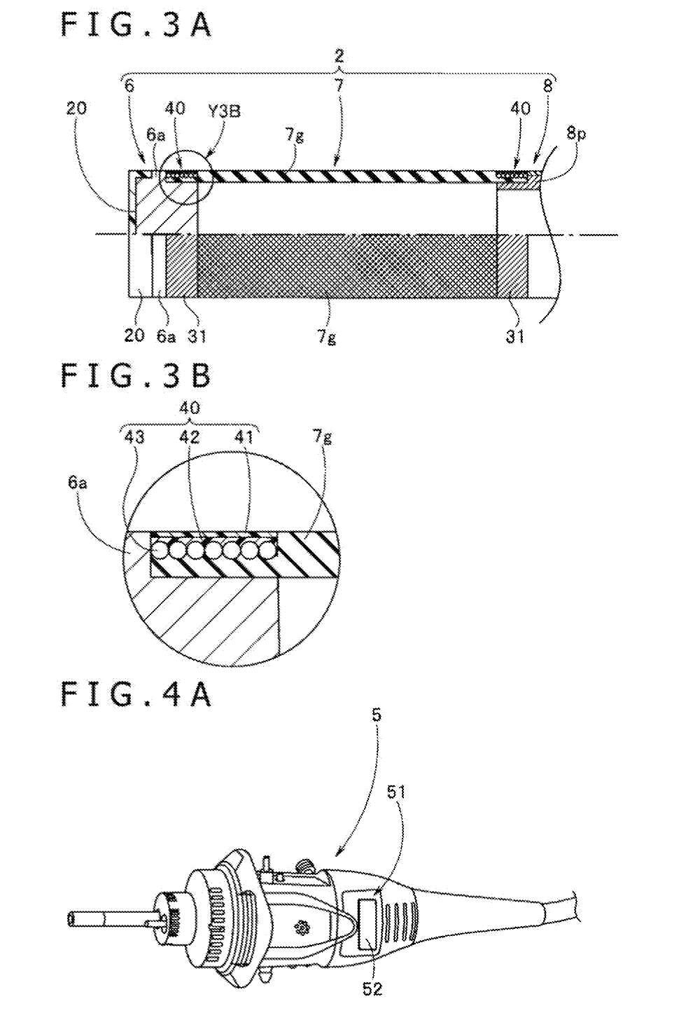

In the embodiment described hereinbefore, the adhesive-securing portion 30 that secures the observation window 21 is of the double-layer structure to give itself the function to determine the deterioration of the adhesive layer. However, as illustrated in FIGS. 3A and 3B, a thread-wound adhesive-securing portion 40 may be of a double-layer structure to give itself the function to determine the deterioration of the adhesive layer.

As illustrated in FIGS. 3A and 3B, the bendable portion 7 includes an outer cladding made of bendable rubber 7g. The bendable rubber 7g is disposed in a predetermined region of a distal-end hard member 6a that is a hard member of the distal-end portion 6 or a predetermined region of a joint tube 8p that is a hard member disposed on a distal-end side of the flexible conduit 8, and is integrally secured in position by the thread-wound adhesive-securing portion 40.

As illustrated in FIG. 3B, the thread-wound adhesive-securing portion 40 includes a first adhesive layer 41 having a surface that is exposed outwardly, a second adhesive layer 42, and a thread-wound portion 43 that are successively arranged from the outside. The thread-wound portion 43 is disposed on an outer circumferential surface of an end portion of the bendable rubber 7g. The thread-wound portion 43 includes a tightening thread pressing down the end portion of the bendable rubber 7g to press an inner circumferential surface of the bendable rubber 7g firmly against an outer circumferential surface of the distal-end hard member 6a. The second adhesive layer 42 is applied to the thread-wound portion 43 and its periphery. The second adhesive layer 42 makes the interface between the hard member and the bendable rubber 7g water-tight. The first adhesive layer 41 is applied to the surface of the second adhesive layer 42 as a display portion and its periphery to cover and conceal the second adhesive layer 42.

As described hereinbefore, the thread-wound adhesive-securing portion 40 that functions as a functional portion for determining the deterioration of the adhesive is disposed on the bendable portion 7 of the endoscope 1. When the endoscope 1 thus arranged is repeatedly cleaned and disinfected, the first adhesive layer 41 disposed in the thread-wound adhesive-securing portion 40 and exposed outwardly is caused to deteriorate. As described hereinbefore, the first adhesive layer 41 is gradually worn away as it deteriorates, and as it is worn away, at least part of the second adhesive layer 42 is exposed through the worn-away area of the first adhesive layer 41. As a consequence, in the same manner as the embodiment described hereinbefore, the user can judge that the first adhesive layer 41 has deteriorated by seeing the second adhesive layer 42, and recognize that the timing to repair the endoscope 1 has come close. As a result, the user is able to reliably send the endoscope 1 for repair without making any errors about the timing to repair the endoscope 1, in the same manner as the embodiment described hereinbefore.

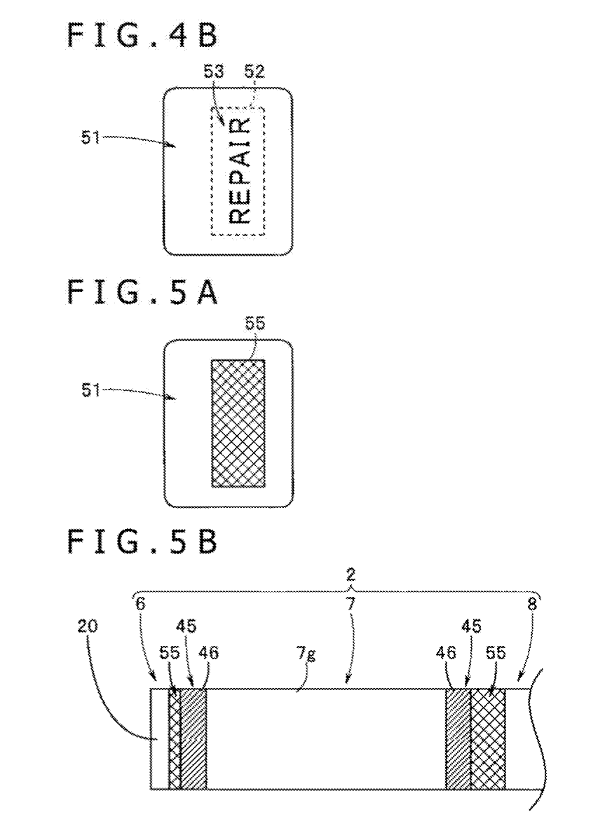

In the embodiment described hereinbefore, the adhesive-securing portion 30 that secures the observation window 21 to the distal-end cover member 20 and the thread-wound adhesive-securing portion 40 that secures the bendable rubber 7g to the hard member have the function to determine the deterioration of the adhesive. When the endoscope 1 is cleaned and disinfected, not only the insertion portion 2, but also the manipulator 3 of the endoscope 1 and the universal cord 4 including the endoscope connector 5 are immersed in an antiseptic solution or exposed to a water steam under high pressure and at high temperature. Therefore, the endoscope connector 5 may have a functional portion to determine the deterioration of the adhesive used in the endoscope 1, as illustrated in FIG. 4A.

As illustrated in FIG. 4A, an adhesive layer 52 that has a surface exposed outwardly is disposed on a predetermined surface 51 of an outer cladding of the endoscope connector 5. The adhesive layer 52 is disposed in covering relation to a display portion to conceal the display portion from external view.

As illustrated in FIG. 4B, the display portion includes a character portion 53. The character portion 53 is disposed on the surface 51 and has its characters covered and concealed in their entirety by the adhesive layer 52. The adhesive layer 52 is formed by applying a non-transparent adhesive. The adhesive layer 52 has a color that is the same as or close to a color of the outer cladding member of the endoscope connector 5.

In contrast, the color of the character portion 53 should preferably be a color that is different from the color of the outer cladding member and is conspicuous and bright against the surroundings. For example, the color of the character portion 53 is a bright orange color. The character portion 53 carries characters “REPAIR” or the like, and is as large in size as possible to make the characters distinguishable with ease.

According to the present embodiment, the adhesive layer 52 is the least resistant to antiseptic solutions or a water steam under high pressure and at high temperature, among the adhesives disposed in the endoscope 1. Therefore, when the endoscope 1 is repeatedly cleaned and disinfected, the adhesive layer 52 is an adhesive portion that is caused to deteriorate earliest among the adhesive portions included in the endoscope 1.

With this arrangement, when the endoscope 1 is repeatedly cleaned and disinfected, the adhesive layer 52 disposed on the surface 51 of the endoscope connector 5 is caused to deteriorate and worn away, making some or all of the characters “REPAIR” of the character portion 53 appear.

The user can judge that the adhesive portion of the endoscope 1 has started to deteriorate by confirming part or all of the character portion 53, and recognize that the timing to repair the endoscope 1 has come close. As a result, the user is able to reliably send the endoscope 1 for repair without making any errors about the timing to repair the endoscope 1, in the same manner as the embodiment described hereinbefore.

With the display portion such as the character portion 53 or the like on the surface 51 of the endoscope connector 5 and the adhesive layer 52 covering and concealing the display portion, the endoscope 1 is given the function to determine the deterioration of the adhesive at a low cost.

The adhesive layer 52 has been exemplified as being the least resistant to antiseptic solutions or a water steam under high pressure and at high temperature, among the adhesives disposed in the endoscope 1. However, the adhesive layer 52 may be the thinnest among the adhesive portions disposed in the endoscope 1. With such an arrangement, when the endoscope 1 is repeatedly cleaned and disinfected, the thinnest adhesive layer 52 is initially caused to deteriorate and worn away, making some or all of the characters “REPAIR” of the character portion 53 appear.

The user now can judge that the adhesive portion of the endoscope 1 has started to deteriorate by confirming the character portion 53, and recognize that the timing to repair the endoscope 1 has come close. The user is thus able to reliably send the endoscope 1 for repair without making any errors about the timing to repair the endoscope 1.

In the embodiment described hereinbefore, the display portion includes the character portion 53 and the character portion 53 carries the characters “REPAIR.” However, the character portion 53 is not limited to the characters “REPAIR,” but may carry other characters or signs or the like indicative of the timing of repair. The characters or signs or the like of the character portion 53 are put in by being directly applied to the surface 51 or by applying a sheet with those characters or signs thereon to the surface 51.

In the embodiment described hereinbefore, moreover, the character portion 53 and the adhesive layer 52 are disposed on the surface 51 of the endoscope connector 5. However, the character portion 53 and the adhesive layer 52 may be disposed on a surface, not illustrated, of the manipulator 3.

In the embodiment described hereinbefore, furthermore, when the first adhesive layer 31 of the adhesive-securing portion 30 or the first adhesive layer 41 of the thread-wound adhesive-securing portion 40 is worn away as it deteriorates, the second adhesive layer 32 or 42 as the display portion appears, indicating the timing to repair the endoscope 1 to the user. However, depending on the type of the adhesive, the adhesive layer may be discolored, rather than being worn away, as it deteriorates. If the adhesive of this type is used, then the adhesive layer is not worn away, but discolored as its deterioration progresses.

In a case where the adhesive-securing portion or the thread-wound adhesive-securing portion is formed using the discoloring adhesive, the adhesive portion includes a single adhesive layer. In this case, as illustrated in FIG. 5A, a determining colored portion 55 of a discolored color is disposed as an indicator on the surface 51 of the endoscope connector 5.

With this arrangement, when the user cleans the endoscope 1, an adhesive-securing portion or a thread-wound adhesive-securing portion, for example, is juxtaposed with the determining colored portion 55, and the color of the surface of the adhesive-securing portion or the thread-wound adhesive-securing portion is compared with a color of the determining colored portion 55 to determine whether they are the same as each other or not.

If it is determined that the color of the adhesive-securing portion or the thread-wound adhesive-securing portion is the same as the color of the determining colored portion 55, then the user judges that the adhesive has deteriorated, and recognizes that the timing to repair the endoscope 1 has come to close. As a result, the user is able to reliably send the endoscope 1 for repair without making any errors about the timing to repair the endoscope 1, in the same manner as the embodiment described hereinbefore.

The determining colored portion 55 is formed by applying an ink, a paint, or the like to the surface 51 of the outer cladding or is formed of a base texture of the surface 51. The determining colored portion 55 may be disposed adjacent to an adhesive layer 46 of a thread-wound adhesive-securing portion 45, as illustrated in FIG. 5B, rather than on the surface 51 of the endoscope connector 5.

This arrangement makes it possible for the user to determine, through an instantaneous visual comparison, whether the adhesive layer 46 of the thread-wound adhesive-securing portion 45 has deteriorated or not. The arrangement also increases the working efficiency by making it unnecessary for the user to visually compare the thread-wound adhesive-securing portion 45 with the determining colored portion 55 on the endoscope connector 5.

According to the embodiment described hereinbefore, the user can judge that the adhesive has deteriorated by seeing the bright orange color or determining that the color of the adhesive is the same as the color of the determining colored portion 55, recognize that the timing to repair the endoscope 1 has come close, and reliably send the endoscope 1 for repair without making any errors about the timing to repair the endoscope 1.

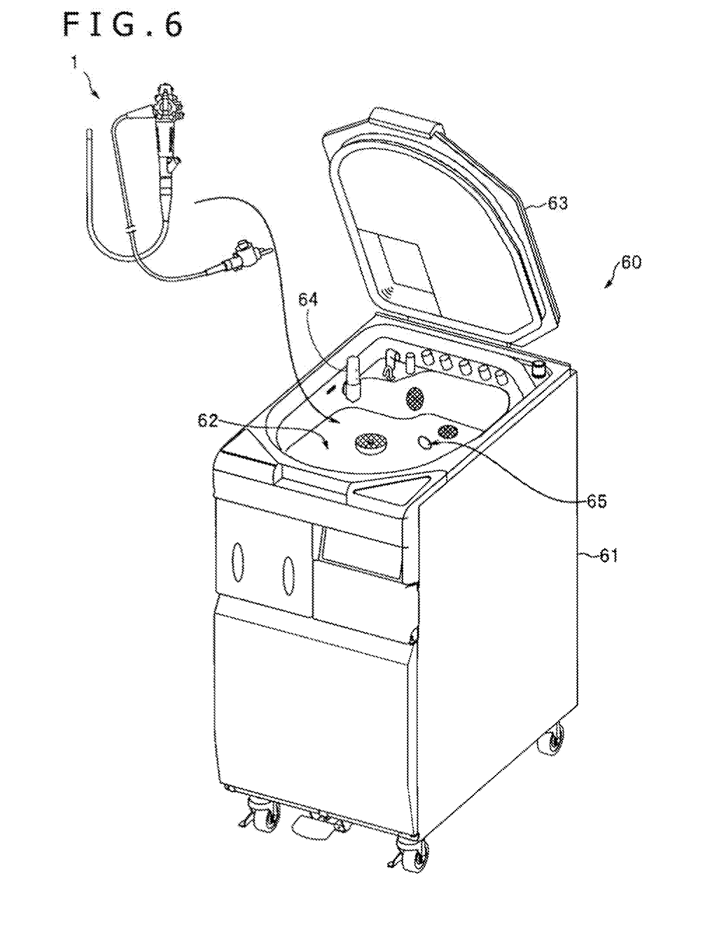

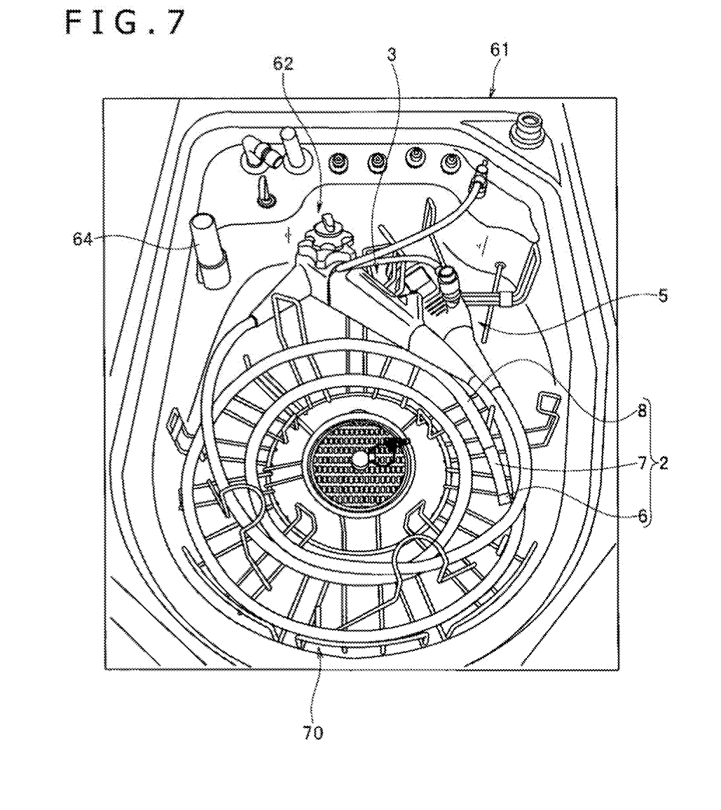

As illustrated in FIG. 6, the endoscope 1 described hereinbefore is cleaned in a cleaning tank 62 disposed in a main apparatus housing 61 of a cleaning and disinfecting apparatus 60. At this time, the endoscope 1 is installed in a predetermined state on an endoscope holding grating (see a numeral reference 70 in FIG. 7), and then is placed together with the endoscope holding grating 70 in a predetermined state in the cleaning tank 62, after which the endoscope 1 is cleaned and disinfected. According to the present embodiment, therefore, the user does not determine the deterioration of the adhesive portion, but the cleaning and disinfecting apparatus 60 determines the deterioration of the adhesive portion.

In FIG. 6, a numeral reference 63 indicates a top cover, a numeral reference 64 a water level sensor, and a numeral reference 65 a determining sensor. The water level sensor 64 detects a level of a liquid supplied to the cleaning tank 62. The determining sensor 65 functions as a reader and includes, for example, a camera optically reading the character portion 53 on the endoscope connector 5.

As illustrated in FIG. 7, in the cleaning tank 62, the endoscope 1 that is installed on the endoscope holding grating 70 has its bendable portion 7 including the distal-end portion 6 of the insertion portion 2, or its manipulator 3, or its endoscope connector 5 disposed in a predetermined position in the cleaning tank 62 of the cleaning and disinfecting apparatus 60. According to the present embodiment, the determining sensor 65 is disposed on a bottom surface of the cleaning tank 62 in confronting relation to the adhesive layer 52 on the surface 51 of the endoscope connector 5.

With this arrangement, while the endoscope 1 is being cleaned and disinfected or after the endoscope 1 has been cleaned and disinfected, if the adhesive layer 52 on the surface 51 of the endoscope connector 5 has deteriorated and has been worn away, causing the orange characters “REPAIR” of the character portion 53 to disappear partly or wholly, then the determining sensor 65 captures an image of the characters “REPAIR” partly or wholly.

The cleaning and disinfecting apparatus 60 has a controller, not illustrated, connected to the determining sensor 65. The controller determines whether there is an orange color in the captured image region from the image captured by the determining sensor 65. If the controller recognizes the orange color, then it determines whether the region of the orange color is in excess of a prescribed value or not.

If the region of the orange color exceeds the prescribed value, then the controller decides that the adhesive layer 52 has deteriorated, and displays “REPAIR,” for example, indicating that the timing to repair the endoscope 1 has come close on a screen on the cleaning and disinfecting apparatus 60. If the region of the orange color is equal to or smaller than the prescribed value, then the controller decides that the adhesive layer 52 has started deteriorating, and displays, for example, “care!” on the screen. As a consequence, the user can accurately grasp the timing to repair the endoscope 1 without having to determine whether the adhesive layer 52 has deteriorated or not, and can reliably send the endoscope 1 for repair while preventing in advance the endoscope 1 from failing due to the deterioration of the adhesive layer 52.

The determining sensor 65 may be placed such that it can capture an image of the adhesive layer 46 of the thread-wound adhesive-securing portion 45 of the insertion portion 2 and the determining colored portion 55. In this case, the controller determines from the image captured by the determining sensor 65 whether the region of the determining colored portion 55 in the captured image region has increased from its original area or not.

Specifically, the controller determines that the adhesive layer 46 has deteriorated if an area representing the sum of the area of the color of the determining colored portion 55 and the area of the discolored color due to the deterioration exceeds a prescribed value, and displays “REPAIR,” for example, indicating that the timing to repair the endoscope 1 has come close on the screen on the cleaning and disinfecting apparatus 60. Consequently, the same operation and advantages as those described hereinbefore are achieved.

In sum, one aspect of the disclosed technology is directed to an endoscope comprises a first adhesive layer disposed on a surface of an outer cladding member and having at least a portion exposed outwardly. A display portion includes a second adhesive layer disposed on a portion of the outer cladding member which is covered with the first adhesive layer. The second adhesive layer having at least a portion which is to be exposed outwardly deteriorates due to frequent cleaning, disinfecting, and/or sterilizing. The second adhesive includes a color different from the first adhesive layer. The display portion is defined by a color that is different from a color of the outer cladding member. The display portion includes characters or signs made of the second adhesive layer.

Another aspect of the disclosed technology is directed to a cleaning and disinfecting apparatus which cleans an endoscope. The endoscope comprises a first adhesive layer disposed on a surface of an outer cladding member and having at least a portion exposed outwardly. A display portion includes a second adhesive layer disposed on a portion of the outer cladding member which is covered with the first adhesive layer. The second adhesive layer having at least a portion which is to be exposed outwardly deteriorates due to frequent cleaning, disinfecting, and/or sterilizing. The second adhesive includes a color different from the first adhesive layer. The cleaning and disinfecting apparatus comprises a reader reading the display portion of the endoscope. The reader optically reads the characters or the signs included in the display portion to detect a state thereof. The reader reads the color of the display portion to detect a state thereof.

A further aspect of the disclosed technology is directed to an endoscope comprises an insertion portion having a proximal end and a distal end. A manipulator is attached to the proximal end of the insertion portion. The insertion portion includes a first adhesive layer exposed outwardly and configured to be deteriorated gradually over time by cleaning, disinfecting, and/or sterilizing. A second adhesive layer covered with the first adhesive layer and having a color different from the first adhesive. The insertion portion includes a cover disposed in the distal end of the insertion portion. The cover includes an observation window formed on a distal-end surface of the cover. An illumination window is formed on the distal-end surface of the cover. The observation window or the illumination window is adhered to the cover by the first adhesive layer and the second adhesive layer.

Yet, a further aspect of the disclosed technology is directed to an endoscope comprises a manipulator and an insertion portion. The insertion portion includes respective proximal and distal ends configured to be attached to the manipulator via the proximal end. The distal end of the insertion layer includes a cover member being mounted thereon. An adhesive-securing portion is used to secure an observation window to the cover portion. The adhesive-securing portion is made of two adhesive layers hardened to one another. The two adhesive layers are defined by respective first and second layers each of which having different color so that when the first adhesive layer begins to deteriorate due to frequent cleaning of the endoscope after usage, the second adhesive layer is exposed to alert a user for maintenance of the endoscope. The second adhesive layer is concealed by the first adhesive layer. The first adhesive layer is black and the second adhesive layer is orange.

The disclosed technology is not limited to only the embodiment described hereinbefore, but various changes and modifications may be made therein without departing from the scope of the invention.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
a first adhesive layer disposed on a surface of an outer cladding member and having at least a portion exposed outwardly; and
a display portion includes a second adhesive layer disposed on a portion of the outer cladding member which is covered with the first adhesive layer, the second adhesive layer having at least a portion which is to be exposed outwardly deteriorates due to frequent cleaning, disinfecting, and/or sterilizing and wherein the second adhesive includes a color different from the first adhesive layer.

2. The endoscope of claim 1, wherein the display portion is defined by a color that is different from a color of the outer cladding member.

3. The endoscope of claim 1, wherein the display portion includes characters or signs made of the second adhesive layer.

4. A cleaning and disinfecting apparatus which cleans an endoscope comprising a first adhesive layer disposed on a surface of an outer cladding member and having at least a portion exposed outwardly and a display portion includes a second adhesive layer disposed on a portion of the outer cladding member which is covered with the first adhesive layer, the second adhesive layer having at least a portion which is to be exposed outwardly deteriorates due to frequent cleaning, disinfecting, and/or sterilizing and wherein the second adhesive includes a color different from the first adhesive layer and wherein the cleaning and disinfecting apparatus comprising:
a reader reading the display portion of the endoscope.

5. The cleaning and disinfecting apparatus of claim 4, wherein the reader optically reads the characters or the signs included in the display portion to detect a state thereof.

6. The cleaning and disinfecting apparatus of claim 4, wherein the reader reads the color of the display portion to detect a state thereof.

7. An endoscope comprising:
an insertion portion having a proximal end and a distal end;
a manipulator attached to the proximal end of the insertion portion;
wherein
the insertion portion includes
a first adhesive layer exposed outwardly and configured to be deteriorated gradually over time by cleaning, disinfecting, or sterilizing,
a second adhesive layer covered with the first adhesive layer and having a color different from the first adhesive.

8. The endoscope of claim 7, wherein the insertion portion includes
a cover disposed in the distal end of the insertion portion, wherein the cover includes
an observation window formed on a distal-end surface of the cover, and
an illumination window formed on the distal-end surface of the cover,
wherein either the observation window or the illumination window is adhered to the cover by the first adhesive layer and the second adhesive layer.

9. An endoscope comprising:
a manipulator; and
an insertion portion having respective proximal and distal ends configured to be attached to the manipulator via the proximal end, the distal end of the insertion layer includes
a cover member being mounted thereon, and
an adhesive-securing portion being used to secure an observation window to the cover portion wherein the adhesive-securing portion is made of two adhesive layers hardened to one another, the two adhesive layers being defined by respective first and second layers each of which having different color so that when the first adhesive layer begins to deteriorate due to frequent cleaning of the endoscope after usage, the second adhesive layer being exposed to alert a user for maintenance of the endoscope.

10. The endoscope of claim 9, wherein the second adhesive layer is concealed by the first adhesive layer.

11. The endoscope of claim 9, wherein the first adhesive layer is black and the second adhesive layer is orange.

12. The endoscope of claim 9 further comprising a cleaning and disinfecting apparatus that is used for cleaning, disinfecting, and/or sterilizing the endoscope.

13. The endoscope of claim 12, wherein the cleaning and disinfecting apparatus includes a reader reading the display portion of the endoscope.

14. The endoscope of claim 13, wherein the reader optically reads the characters or the signs included in the display portion to detect a state thereof.

15. The endoscope of claim 13, the reader reads the color of the display portion to detect a state thereof.
